(19) United States
(12) Patent Application Publication
(10) Pub. No.: US 2026/0306393 A1
(43) Pub. Date: Oct. 8, 2026

(54) COMPOSITION COMPRISING LOW MOLECULAR WEIGHT CHITOSAN AND GLYCOLIPIDS

(71) Applicant: L'OREAL, PARIS (FR)

(72) Inventors: Shinsuke OKUDA, Kawasaki-shi (JP); Takehiko KASAI, Kawasaki-shi (JP); Tatsushi ISOJIMA, Kawasaki-shi (JP); Mariko YAMAMOTO, Kawasaki-shi (JP)

(21) Appl. No.: 19/485,790

(22) PCT Filed: May 14, 2024
(86) PCT No.: PCT/JP2024/080070

§ 371 Date: Nov. 19, 2025

(30) Foreign Application Priority Data
Jun. 20, 2023 (JP) 2023-101054
Jul. 24, 2023 (FR) FR2307943

(51) Int. Cl. A61K 8/73; A61K 8/60; A61Q 19/10

(52) U.S. Cl. CPC A61K 8/736; A61K 8/602; A61Q 19/10; A61K 2800/10

(57) ABSTRACT

A composition contains: (a) at least one cationic polymer selected from chitosans; and (b) at least one glycolipid. The (a) cationic polymer has a molecular weight of less than 20,000, preferably less than 15,000 and more preferably less than 10,000. The composition can provide a keratin substance such as skin with a deposition including glycolipids. In addition, the composition can include an environmentally-friendly ingredient.

### TECHNICAL FIELD

The present invention relates to a composition including a low molecular weight chitosan and glycolipids, as well as a cosmetic process using the composition and a use relating to the composition.

### BACKGROUND ART

The formulation of environmentally-friendly cosmetic products, which are designed and developed considering environmental issues, is becoming a major goal in an effort to meet global challenges.

It is therefore essential to propose more sustainable compositions, preparation processes and ingredients to address these environmental concerns.

In this context, it is important to develop new cosmetic compositions with a better carbon footprint, particularly by promoting the use of renewable raw materials and/or materials with a good index of naturalness and/or materials of natural origin and, more particularly, materials of plant origin while reducing the use of compounds of petrochemical origin.

Glycolipids such as rhamnolipids are known to have unique bioactivities, such as anti-inflammation efficacy, anti-allergic efficacy, antibacterial efficacy, and the like.

WO 2020/178048 discloses the use of glycolipids as a deposition aid for some substances. However, WO 2020/178048 is silent about a low molecular weight chitosan.

### DISCLOSURE OF INVENTION

There is a need for a composition which can provide a keratin substance such as skin with a deposition including glycolipids.

Thus, a first objective of the present invention is to provide a composition which can provide a keratin substance such as skin with a deposition including glycolipids.

In addition, a second objective of the present invention is to provide a composition which can include at least one environmentally-friendly ingredient.

The above objectives of the present invention can be achieved by a composition, preferably a cosmetic composition, and more preferably a cosmetic composition for a keratin substance such as skin, comprising:
(a) at least one cationic polymer selected from chitosans; and
(b) at least one glycolipid,
wherein
the (a) cationic polymer has a molecular weight of less than 20,000, preferably less than 15,000 and more preferably less than 10,000.

The amount of the (a) cationic polymer(s) in the composition according to the present invention may be from 0.01% to 15% by weight, preferably from 0.05% to 10% by weight, and more preferably from 0.1% to 5% by weight, relative to the total weight of the composition.

The (b) glycolipid may be selected from rhamnolipids.

The amount of the (b) glycolipid(s) in the composition according to the present invention may be from 0.01% to 15% by weight, preferably from 0.1% to 10% by weight, and more preferably from 1% to 5% by weight, relative to the total weight of the composition.

The composition according to the present invention may further comprise (c) water.

The composition according to the present invention may further comprise (d) at least one amphoteric surfactant.

The amount of the (d) amphoteric surfactant(s) in the composition according to the present invention may be from 0.01% to 20% by weight, preferably from 0.1% to 15% by weight, and more preferably from 1% to 10% by weight, relative to the total weight of the composition.

The composition according to the present invention may further comprise (e) at least one amino acid surfactant.

The amount of the (e) amino acid surfactant(s) in the composition according to the present invention may be from 0.01% to 20% by weight, preferably from 0.1% to 15% by weight, and more preferably from 1% to 10% by weight, relative to the total weight of the composition.

The composition according to the present invention may further comprise (f) at least one monovalent non-polymeric acid or a salt thereof, preferably a monovalent non-polymeric carboxylic acid, and more preferably a monovalent hydroxy acid such as lactic acid and salicylic acid.

The amount of the (f) monovalent non-polymeric acid or a salt thereof in the composition according to the present invention may be from 0.01% to 15% by weight, preferably from 0.05% to 10% by weight, and more preferably from 0.1% to 5% by weight, relative to the total weight of the composition.

The pH of the composition according to the present invention may be from 4 to 8, preferably 4.5 to 7.5, and more preferably from 5 to 7.

The composition according to the present invention may be a cleansing composition, preferably a cleansing composition for skin, and more preferably a cleansing composition for face.

The present invention also relates to a cosmetic process for a keratin substance such as skin, comprising:
applying to the keratin substance the composition according to the present invention; and
optionally removing the composition from the keratin substance.

The present invention also relates to a use of (a) at least one cationic polymer selected from chitosans with a molecular weight of less than 20,000, preferably less than 15,000 and more preferably less than 10,000,
in a composition comprising
(b) at least one glycolipid,
in order to provide precipitates including the (b) glycolipid when diluting the composition with water.

### BEST MODE FOR CARRYING OUT THE INVENTION

After diligent research, the inventors have discovered that it is possible to provide a composition which can provide a keratin substance such as skin with a deposition including glycolipids, and can include at least one environmentally-friendly ingredient.

Thus, the composition according to the present invention comprises:
(a) at least one cationic polymer selected from chitosans; and
(b) at least one glycolipid,
wherein
the (a) cationic polymer has a molecular weight of less than 20,000, preferably less than 15,000 and more preferably less than 10,000.

The composition according to the present invention can provide a keratin substance such as skin with a deposition including glycolipids. The deposition can be provided by, for example, precipitates including glycolipids formed in the composition according to the present invention.

Furthermore, a preferable embodiment of the composition according to the present invention can provide an increased amount of deposition or precipitates to a keratin substance such as skin.

As glycolipids such as rhamnolipids have bioactivities, such as anti-inflammation efficacy, anti-allergic efficacy, and antibacterial efficacy, the deposition including the (b) glycolipid provided by the composition according to the present invention is useful to care for a keratin substance such as skin. In particular, the composition according to the present invention is useful to treat or care for skin with acne.

Furthermore, since glycolipids can function as surfactants, the deposition including the (b) glycolipid can provide enhanced cleansing effects. Thus, the composition according to the present invention is also useful for cleansing a keratin substance such as skin.

Since chitosans can be obtained from natural resources, the (a) cationic polymer is an environmentally-friendly ingredient. Therefore, the composition according to the present invention can include an environmentally-friendly ingredient. In addition, the (b) glycolipid can be originated from renewable materials such as biodegradable materials. Therefore, the composition according to the present invention can be environmentally-friendly.

Hereinafter, the present invention will be explained in a more detailed manner.

### [Composition]

### (Cationic Polymer)

The composition according to the present invention comprises (a) at least one cationic polymer. A single type of cationic polymer may be used, or two or more different types of cationic polymers may be used in combination.

A cationic polymer has a positive charge density. The charge density of the (a) cationic polymer may be from 0.01 meq/g to 20 meq/g, preferably from 0.05 to 15 meq/g, and more preferably from 0.1 to 10 meq/g.

According to the present invention, the (a) cationic polymer is selected from chitosans. Two or more chitosans may be used in combination.

It is preferable that the (a) cationic polymer be selected from chitosans.

The molecular weight (Da) of the (a) cationic polymer is less than 20,000, preferably less than 15,000, and more preferably less than 10,000. In other words, the (a) cationic polymer is a low molecular weight chitosan.

The molecular weight (Da) of the (a) cationic polymer may be more than 1,000, preferably more than 1,500, and more preferably more than 2,000.

Thus, the molecular weight (Da) of the (a) cationic polymer may be more than 1,000 and less than 20,000, preferably more than 1,500 and less than 15,000, and more preferably more than 2,000 and less than 10,000.

Unless otherwise defined in the descriptions, “molecular weight” means a weight average molecular weight. The molecular weight can be measured or determined by a gel permeation chromatography, for example, in accordance with ASTM D5296-19.

Chitosan is very uncommon in nature. It is only reported in the exoskeletons of certain insects such as termite queens and in the cell walls of a particular class of fungi, zygomycetes.

Chitosan may be obtained by deacetylation of chitin. Chitin is a polysaccharide composed of several N-acetyl-D-glucosamine units linked together by a type β bond (1,4).

The ideal chemical structure of chitosan is a sequence of β-D-glucosamine monomers connected by a glycosidic bond (1→4).

“Chitosan” according to the present invention means any copolymer formed of constituent units N-acetyl-D-glucosamine and D-glucosamine, whose degree of acetylation is less than 90%, preferably less than 80%, preferably less than 70%, preferably less than 60%, preferably less than 50%. Chitosan consists of glucosamine sugar units (deacetylated units) and N-acetyl-D-glucosamine units (acetylated units) linked together by β type bonds (1,4) and is a polymer of the Poly (N-acetyl-D-glucosamine)-poly (D-glucosamine) type.

More preferably, the degree of acetylation of chitosan is less than or equal to 40%, preferably less than or equal to 35%, preferably less than or equal to 25%, preferably less than or equal to 15%, and preferably less than or equal to 10%.

The degree of acetylation is the percentage of acetylated units relative to the number of total units, it can be determined by Fourier transform infrared spectroscopy (FT-IR) or by titration by a strong base.

The chitosan of the present invention is preferably a polysaccharide prepared from a fungal origin. In particular, it is extracted and purified from safe and abundant food or biotechnological fungal sources such as Agaricus bisporus or Aspergillus niger.

The chitosan of the present invention is preferably derived from the mycelium of a fungus of the Ascomycete type, and in particular Aspergillus niger and/or a Basidiomycete fungus, and in particular Lentinula edodes (shiitake) and/or Agaricus bisporus. Preferably the fungus is Aspergillus niger.

Chitosan may be of GMO (Genetically Modified Organisms) origin, but preferably is of non-GMO origin.

The chitosan according to the present invention is native, that is to say that it is not modified. In particular, it does not contain any chemical modification.

One method of preparing chitosan is that described in WO03/068824.

Preferably, the chitosan used in the present invention is in a powder form. It is marketed by Glentham Life Science under the name GU3511.

The amount of the (a) cationic polymer(s) in the composition according to the present invention may be 0.01% by weight or more, preferably 0.05% by weight or more, and more preferably 0.1% by weight or more, relative to the total weight of the composition. It may be even more preferable that the amount of the (a) cationic polymer be 1% by weight or more, relative to the total weight of the composition.

The amount of the (a) cationic polymer(s) in the composition according to the present invention may be 15% by weight or less, preferably 10% by weight or less, and more preferably 5% by weight or less, relative to the total weight of the composition.

The amount of the (a) cationic polymer(s) in the composition according to the present invention may be from 0.01% to 15% by weight, preferably from 0.05% to 10% by weight, and more preferably from 0.1% to 5% by weight, relative to the total weight of the composition. It may be even more preferable that the amount of the (a) cationic polymer be from 1% to 5% by weight, relative to the total weight of the composition.

### (Glycolipid)

The composition according to the present invention comprises (b) at least one glycolipid. A single type of glycolipid may be used, or two or more different types of glycolipids may be used in combination.

The term “glycolipid” is understood as meaning a compound formed from a lipid to which are attached one or more sugar compounds.

The (b) glycolipid may be selected from glucolipids, sophorolipids, trehalolipids, cellobiose lipids, rhamnolipids, and mixtures thereof.

The (b) glycolipid may preferably be selected from sophorolipids rhamnolipids, and mixtures thereof, and more preferably from rhamnolipids.

### Glucolipids:

The (b) glycolipid may be selected from glucolipids, which contain a glucose moiety and can be represented by the general formula (I):

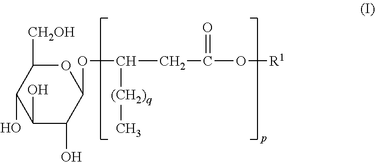

in which:
R¹ represents a hydrogen atom or a cation,
p denotes an integer ranging from 1 to 4, and
q denotes an integer ranging from 4 to 10, preferably equal to 6.

The glucolipids can be produced by the bacterium Alcaligenes sp. MM1.

Appropriate fermentation methods are reviewed by M. Schmidt in his doctoral thesis (1990), Technical University of Braunschweig, and by Schulz et al. (1991) Z. Naturforsch., 46C, 197-203. The glucolipids are recovered from the fermentation broth by solvent extraction using diethyl ether or a dichloromethane:methanol or chloroform:methanol mixture.

### Sophorolipids:

The (b) glycolipid may be selected from sophorolipids, which contain a sophorose moiety and can be represented by the general formula (II):

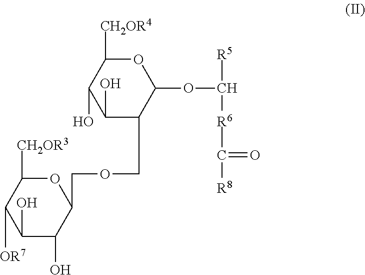

in which:
R³ and R⁴ individually represent a hydrogen atom or an acetyl group,
R⁵ represents a saturated or unsaturated, hydroxylated or non-hydroxylated hydrocarbon group having from 1 to 9 carbon atoms, preferably methyl,
R⁶ represents a saturated or unsaturated, hydroxylated or non-hydroxylated hydrocarbon group having from 1 to 19 carbon atoms,
   with the proviso that the total number of carbon atoms in the groups R⁵ and R⁶ does not exceed 20 and is preferably from 14 to 18.

Sophorolipids may be incorporated into the composition according to the present invention either in the form of an open-chain free acid, where R⁷ represents a hydrogen atom and R⁸ represents a hydroxy group OH, or in its lactone form, where a lactone ring is formed between R⁷ and R⁸, as indicated by formula (III):

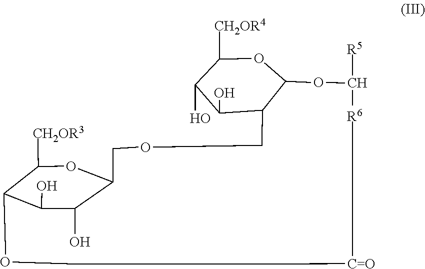

in which:
R³, R⁴, R⁵ and R⁶ are as defined above,
   with the proviso that at least one of R³ and R⁴ represents an acetyl group.

The sophorolipids can be produced by yeast cells, for example, Torulopsis apicola and Torulopsis bombicola cells. The fermentation process generally uses sugars and alkanes as substrates.

Appropriate fermentation methods are reviewed in A. P. Tulloch, J. F. T. Spencer and P. A. J. Gorin, Can. J. Chem. (1962), 40, 1326, and U. Gobbert, S. Lang and F. Wagner, Biotechnology Letters (1984), 6 (4), 225. The resulting product is a mixture of various open-chain sophorolipids and of sophorolipid lactones that may be used in the form of mixtures, or the required form may be isolated.

It is possible to use as a sophorolipid, for example that sold under the name Sopholiance S by Givaudan and that sold under the name BioToLife by BASF.

### Trehalolipids:

The (b) glycolipid may be selected from trehalolipids, which contain a trehalose fragment and can be represented by the general formula (IV):

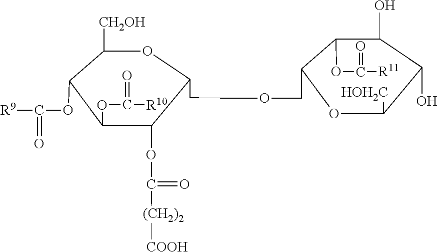

in which:
R⁹, R¹⁰ and R¹¹ individually represent a saturated or unsaturated, hydroxylated or non-hydroxylated hydrocarbon radical having from 5 to 13 carbon atoms.

The trehalolipids can be produced by bacterial fermentation using the marine bacterium Arthrobacter sp. Ek 1 or the freshwater bacterium Rhodococcus erythropolis. Appropriate fermentation methods are provided by Ishigami et al. (1987), J. Jpn. Oil Chem. Soc., 36, 847-851, Schultz et al. (1991), Z. Naturforsch., 46C, 197-203, and Passeri et al. (1991), Z. Naturforsch., 46C, 204-209.

### Cellobiose Lipids:

The (b) glycolipid may be selected from cellobiose lipids, which contain a cellobiose fragment and can be represented by the general formula (V):

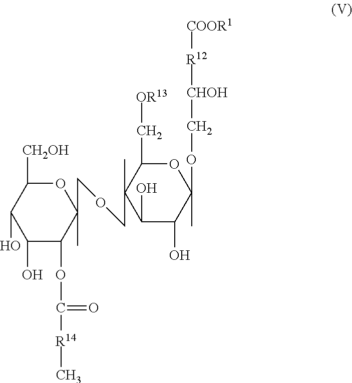

in which:
R¹ represents a hydrogen atom or a cation,
R¹² represents a saturated or unsaturated, hydroxylated or non-hydroxylated hydrocarbon radical having from 9 to 15 carbon atoms, preferably 13 carbon atoms,
R¹³ represents a hydrogen atom or an acetyl group; and
R¹⁴ represents a saturated or unsaturated, hydroxylated or non-hydroxylated hydrocarbon radical having from 4 to 16 carbon atoms.

The cellobiose lipids can be produced by cells of fungi of the genus Ustilago. Appropriate fermentation processes are provided by Frautz, Lang and Wagner (1986), Biotech. Letts., 8, 757-762.

### Rhamnolipids:

The (b) glycolipids may be selected from rhamnolipids.

The composition according to the present invention preferably comprises one or more rhamnolipids.

Rhamnolipids are glycolipids produced by various bacterial species. They consist of one rhamnose fragment (mono-rhamnolipid) or of two rhamnose fragments (di-rhamnolipid) linked by a glycosidic bond to one, two or three chains of β-hydroxylated fatty acids linked to one another by an ester bond.

More specifically, these mono-rhamnolipids and di-rhamnolipids correspond to the following formula (VI):

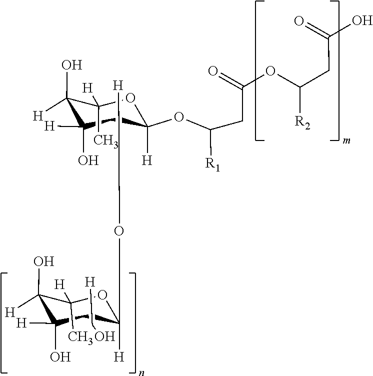

in which:
m denotes an integer equal to 2, 1 or 0,
n denotes an integer equal to 1 or 0, and
R₁ and R₂, each independently represent identical or different hydrocarbon radicals having from 2 to 24 carbon atoms, preferably from 5 to 13 carbon atoms, that are branched or unbranched, substituted or unsubstituted, in particular hydroxy-substituted, and saturated or unsaturated, preferably a singly, doubly or triply unsaturated alkyl radical.

Thus, when n is equal to 0, the formula (VI) protects mono-rhamnolipids and, when n is equal to 1, it protects di-rhamnolipids.

The composition according to the present invention preferably comprises at least one di-rhamnolipid.

The composition according to the present invention preferably comprises at least one di-rhamnolipid of formula (VI) in which:
m denotes an integer equal to 2, 1 or 0;
n denotes an integer equal to 1; and
R₁ and R₂, each independently represent identical or different hydrocarbon radicals having from 2 to 24 carbon atoms, preferably from 5 to 13 carbon atoms, that are branched or unbranched, substituted or unsubstituted, in particular hydroxy-substituted, and saturated or unsaturated, preferably a singly, doubly or triply unsaturated alkyl radical, and also the salts thereof, solvates thereof and optical isomers thereof.

The glycosidic bond between the two rhamnose fragments may be in the alpha or beta configuration and is preferably in the alpha configuration.

In the context of the present invention,
the salts of the di-rhamnolipids of formula (VI) are more particularly the carboxylate salts thereof with an organic or inorganic cation and especially with a cation selected from sodium, potassium, calcium and ammonium.
the solvated forms of the di-rhamnolipids of formula (VI) are more particularly those solvated with one or more molecules of water or of organic solvents, for example a hydrate or a solvate of a linear or branched alcohol, such as ethanol or isopropanol, the optically active carbon atoms of the fatty acids preferably being in the form of the R enantiomers, and
the term “alkyl” radical denotes a saturated, linear or branched aliphatic group; for example, a C₁-C₂₀ alkyl group having a linear or branched hydrocarbon chain of 1 to 20 carbon atoms, more particularly a methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl or eicosyl.

The composition according to the present invention preferably comprises at least one di-rhamnolipid of formula (VI) in which:
m denotes an integer equal to 2, 1 or 0;
n denotes an integer equal to 1; and
R₁ and R₂, which are identical or different, are selected from pentenyl, hexenyl, heptenyl, octenyl, nonenyl, decenyl, undecenyl, dodecenyl and tridecenyl radicals and radicals of formula —(CH₂)ₒCH₃, with o denoting an integer ranging from 1 to 23, in particular from 3 to 15 and more particularly from 4 to 12.

According to one embodiment of the present invention, the composition according to the present invention comprises at least one di-rhamnolipids of general formula (VI) in which m is equal to 1

According to one embodiment of the present invention, the composition according to the present invention comprises a mixture of at least two, preferably at least three, di-rhamnolipids of general formula (VI) in which m is preferably equal to 1.

According to another embodiment of the present invention, the composition according to the present invention comprises a mixture comprising at least one mono-rhamnolipid.

More preferably, the composition according to the present invention comprises at least one dirhamnolipid of the following formula (VII):

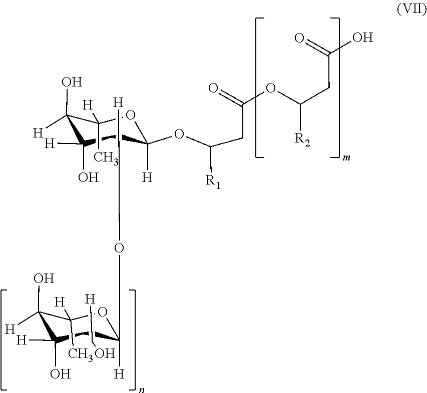

in which:
m denotes an integer equal to 2, 1 or 0; preferably, m is equal to 1,
n denotes an integer equal to 1,
R₁ is a —(CH₂)ₚ—CH₃ radical, with p being an integer varying from 1 to 23, preferably from 4 to 12,
R₂ is a —(CH₂)_{q}—CH₃ radical, with q being an integer varying from 1 to 23, preferably from 4 to 12,
   and also the salts thereof, solvates thereof and optical isomers thereof.

By way of illustration and without limiting the di-rhamnolipids of formula (VII) that may be suitable for the present invention, mention may be made in particular of the compounds of formula di-RL-CXCY, such as are defined in Table 1 below.

The formula di-RL-CXCY is an alternative way of writing in order to represent a di-rhamnolipid (di-RL) functionalized by two radicals R₁ and R₂ respectively represented by the symbols CX and CY, the integers X and Y being respectively equal to p+4 and q+4.

TABLE 1

| Composés | Di-RL-CXCY | p | q |
|---|---|---|---|
| 1 | diRL-C8C8 | 4 | 4 |
| 2 | diRL-C8C10 | 4 | 6 |
| 3 | diRL-C10C8 | 6 | 4 |
| 4 | diRL-C10C10 | 6 | 6 |
| 5 | diRL-C10C12 | 6 | 8 |
| 6 | diRL-C12C10 | 8 | 6 |
| 7 | diRL-C12C12 | 8 | 8 |
| 8 | diRL-C12C14 | 8 | 10 |
| 9 | diRL-C14C12 | 10 | 8 |
| 10 | diRL-C14C14 | 10 | 10 |
| 11 | diRL-C14C16 | 10 | 12 |
| 12 | diRL-C16C14 | 12 | 10 |
| 13 | diRL-C16C16 | 12 | 12 |

According to a preferred embodiment, the composition according to the present invention comprises at least one di-rhamnolipid of formula (VII) in which p and q are identical and equal to 6 and m is equal to 1, also referred to as di-RL-C10C10, or one of the salts, solvates and optical isomers thereof.

Preferably, the di-rhamnolipid of formula (VII) in which p and q are identical and equal to 6 and m is equal to 1 is present in the composition according to the present invention in a proportion of at least 50% by weight and preferably of from 51% to 85% by weight, relative to the total weight of rhamnolipids.

According to another embodiment, the composition according to the present invention comprises at least one di-rhamnolipid of formula (VII) in which m is equal to 1, p is equal to 6 and q is equal to 8.

According to another embodiment, the composition according to the present invention comprises at least one di-rhamnolipid of formula (VI) in which n and m are equal to 1, R₁ represents a —(CH₂)ₒCH₃ radical, with o being an integer varying from 4 to 12, and R₂ is selected from the pentenyl, hexenyl, heptenyl, octenyl, nonenyl, decenyl, undecenyl, dodecenyl and tridecenyl radicals; preferably, R₁ represents a —(CH₂)₆CH₃ radical and R₂ a nonenyl radical.

According to another preferred embodiment, the composition according to the present invention comprises a mixture of at least two, in particular at least three, di-rhamnolipids of formula (VI) or of formula (VII) selected from:
a di-rhamnolipid of formula (VII) in which p and q are identical and equal to 6 and m is equal to 1;
a di-rhamnolipid of formula (VII) in which m is equal to 1, p is equal to 6 and q is equal to 8; and
at least one di-rhamnolipid of formula (VI) in which n and m are equal to 1, R₁ represents a —(CH₂)ₒCH₃ radical, with o being an integer varying from 4 to 12, and R₂ is selected from the pentenyl, hexenyl, heptenyl, octenyl, nonenyl, decenyl, undecenyl, dodecenyl and tridecenyl radicals; preferably, R₁ represents a —(CH₂)₆CH₃ radical and R₂ a nonenyl radical.

Preferably, the composition according to the present invention comprises a mixture of at least two, in particular at least three, di-rhamnolipids of formula (VI) or of formula (VII) selected from:
at least 50% by weight and preferably of from 51% to 85% by weight of a di-rhamnolipid of formula (VII) in which p and q are identical and equal to 6 and m is equal to 1, relative to the total weight of rhamnolipids,
from 0.5% to 25% by weight, preferably from 5% to 15% by weight, of a dirhamnolipid of formula (VII) in which p is equal to 6, q is equal to 8 and m is equal to 1, relative to the total weight of rhamnolipids, and
from 0.5% to 15% by weight, preferably from 3% to 12% by weight, preferably from 5% to 10% by weight, of a dirhamnolipid of formula (VI) in which n and m are equal to 1, R₁ represents a —(CH₂)₆CH₃ radical and R₂ represents a nonenyl radical, relative to the total weight of rhamnolipids.

As specified above, rhamnolipids are customarily prepared by processes known to those skilled in the art starting from bacterial producers, such as Pseudomonas.

Appropriate fermentation methods are reviewed by D. Haferburg, R. Hommel, R. Claus and H. P. Kleber in Adv. Biochem. Ing./Biotechnol. (1986), 33, 53-90, and by F. Wagner, H. Bock and A. Kretschmar in Fermentation (ed. R. M. Lafferty) (1981), 181-192, Springer Verlag, Vienna.

Use may be made, as rhamnolipid, of the one sold under the name Rheance One by Evonik (INCI name: glycolipids).

The amount of the (b) glycolipid(s) in the composition according to the present invention may be 0.01% by weight or more, preferably 0.1% by weight or more, and more preferably 1% by weight or more, relative to the total weight of the composition.

The amount of the (b) glycolipid(s) in the composition according to the present invention may be 15% by weight or less, preferably 10% by weight or less, and more preferably 5% by 35 weight or less, relative to the total weight of the composition.

The amount of the (b) glycolipid(s) in the composition according to the present invention may be from 0.01% to 15% by weight, preferably from 0.1% to 10% by weight, and more preferably from 1% to 5% by weight, relative to the total weight of the composition.

### (Water)

The composition according to the present invention may comprise (c) water.

The amount of the (c) water in the composition according to the present invention may be 50% by weight or more, preferably 60% by weight or more, and more preferably 70% by weight or more, relative to the total weight of the composition.

The amount of the (c) water in the composition according to the present invention may be 99% by weight or less, preferably 97% by weight or less, and more preferably 95% by weight or less, relative to the total weight of the composition.

The amount of the (c) water in the composition according to the present invention may be from 50% to 99% by weight, preferably from 60% to 97% by weight, and more preferably from 70% to 95% by weight, relative to the total weight of the composition.

### (Amphoteric Surfactant)

The composition according to the present invention may comprise (d) at least one amphoteric surfactant. A single type of amphoteric surfactant may be used, or two or more different types of amphoteric surfactants may be used in combination.

The amphoteric or zwitterionic surfactants can be, for example (non-limiting list), amine derivatives such as aliphatic secondary or tertiary amine, and optionally quaternized amine derivatives, in which the aliphatic radical is a linear or branched chain including 8 to 22 carbon atoms and containing at least one water-solubilizing anionic group (for example, carboxylate, sulphonate, sulphate, phosphate or phosphonate).

The (d) amphoteric surfactant may preferably be selected from the group consisting of betaines and amidoaminecarboxylated derivatives.

It is preferable that the (d) amphoteric surfactant be selected from betaine-type surfactants.

The betaine-type amphoteric surfactant is preferably selected from the group consisting of alkylbetaines, alkylamidoalkylbetaines, sulfobetaines, alkylsulfobetaines, phosphobetaines, alkylphosphobetaines, and alkylamidoalkylsulfobetaines, in particular, (C₈-C₂₄)alkylbetaines, (C₈-C₂₄)alkylamido(C₁-C₈)alkylbetaines, sulfobetaines, (C₁-C₈)alkylsulfobetaines, phosphobetaines, (C₁-C₈)alkylphosphobetaines, and (C₅-C₂₄)alkylamido(C₁-C₈)alkylsulfobetaines. In one embodiment, the amphoteric surfactants of betaine type are chosen from (C₈-C₂₄)alkylbetaines, (C₈-C₂₄)alkylamido(C₁-C₈)alkylsulfobetaines, sulfobetaines, (C₁-C₈)alkylsulfobetaines and phosphobetaines.

Non-limiting examples that may be mentioned include the compounds classified in the CTFA International Cosmetic Ingredient Dictionary & Handbook, 15th Edition, 2014, under the names cocobetaine, laurylbetaine, cetylbetaine, coco/oleamidopropylbetaine, cocamidopropylbetaine, palmitamidopropylbetaine, stearamidopropylbetaine, cocamidoethylbetaine, cocamidopropylhydroxysultaine, oleamidopropylhydroxysultaine, cocohydroxysultaine, laurylhydroxysultaine, and cocosultaine, alone or as mixtures.

The betaine-type amphoteric surfactant (betaines) is preferably an alkylbetaine an alkylsulfobetaine, and an alkylamidoalkylbetaine, in particular cocobetaine, sulfopropylbetaine, and cocamidopropylbetaine.

Among the amidoaminecarboxylated derivatives, mention may be made of the products sold under the name Miranol, as described in U.S. Pat. Nos. 2,528,378 and 2,781,354 and classified in the CTFA dictionary, 3rd edition, 1982 (the disclosures of which are incorporated herein by reference), under the names Amphocarboxyglycinates and Amphocarboxypropionates, with the respective structures:

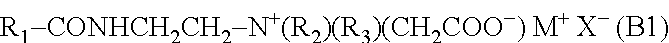

in which:
R₁ denotes an alkyl radical of an acid R₁—COOH present in hydrolysed coconut oil, a heptyl, nonyl or undecyl radical,
R₂ denotes a beta-hydroxyethyl group,
R₃ denotes a carboxymethyl group,
M⁺ denotes a cationic ion derived from alkaline metals such as sodium; ammonium ion; or an ion derived from an organic amine;
X⁻ denotes an organic or inorganic anionic ion such as halides, acetates, phosphates, nitrates, alkyl(C₁-C₄)sulfates, alkyl(C₁-C₄)— or alkyl(C₁-C₄)aryl-sulfonates, particularly methylsulfate and ethylsulfate; or M⁺ and X⁻ are not present;

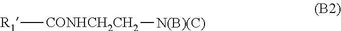

in which:
R₁′ denotes an alkyl radical of an acid R₁′—COOH present in coconut oil or in hydrolysed linseed oil, an alkyl radical, such as a C₇, C₉, C₁₁ or C₁₃ alkyl radical, a C₁₇ alkyl radical and its iso-form, or an unsaturated C₁₇ radical,
B represents —CH₂CH₂OX′,
C represents —(CH₂)_{z}—Y′, with z=1 or 2,
X′ denotes a —CH₂—COOH group, —CH₂—COOZ′, —CH₂CH₂—COOH, —CH₂CH₂—COOZ′ or a hydrogen atom, and
Y′ denotes —COOH, —COOZ′, —CH₂—CHOH—SO₃Z′, —CH₂—CHOH—SO₃H radical or a —CH₂—CH(OH)—SO₃—Z′ radical,
wherein Z′ represents an ion of an alkaline or alkaline earth metal such as sodium, an ion derived from an organic amine or an ammonium ion;
and

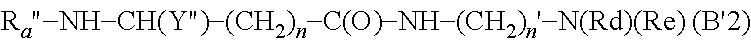

in which:
Y″ denotes —C(O)OH, —C(O)OZ″, —CH₂—CH(OH)—SO₃H or —CH₂—CH(OH)—SO₃—Z″, wherein
Z″ denotes a cationic ion derived from alkaline metal or alkaline-earth metals such as sodium, an ion derived from organic amine or an ammonium ion;
Rd and Re denote a C₁-C₄ alkyl or C₁-C₄ hydroxyalkyl radical;
R_{a″} denotes a C₁₀-C₃₀ group alkyl or alkenyl group from an acid, and
n and n′ independently denote an integer from 1 to 3.

It is preferable that the (d) amphoteric surfactant with formula B1 and B2 be selected from (C₈-C₂₄)-alkyl amphomonoacetates, (C₈-C₂₄)alkyl amphodiacetates, (C₈-C₂₄)alkyl amphomonopropionates, and (C₈-C₂₄)alkyl amphodipropionates

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphopropionate, Disodium Caprylamphodipropionate, Disodium Caprylamphodipropionate, Lauroamphodipropionic acid and Cocoamphodipropionic acid.

By way of example, mention may be made of the cocoamphodiacetate sold under the trade name Miranol® C2M concentrate by the company Rhodia Chimie.

Among compounds of formula (B′2), mention may be made of sodium diethylaminopropyl cocoaspartamide (CTFA) marketed by CHIMEX under the denomination CHIMEXANE HB.

It is preferable that the (d) amphoteric surfactant be selected from betaine-type surfactants, more preferably from alkylbetaines, alkylamidoalkylbetaines, and mixtures thereof, and even more preferably selected from cocobetaine, cocamidopropylbetaine, and a mixture thereof.

The amount of the (d) amphoteric surfactant(s) in the composition according to the present invention may be 0.01% by weight or more, preferably 0.1% by weight or more, and more preferably 1% by weight or more, relative to the total weight of the composition.

The amount of the (d) amphoteric surfactant(s) in the composition according to the present invention may be 20% by weight or less, preferably 15% by weight or less, and more preferably 10% by weight or less, relative to the total weight of the composition.

The amount of the (d) amphoteric surfactant(s) in the composition according to the present invention may be from 0.01% to 20% by weight, preferably from 0.1% to 15% by weight, and more preferably from 1% to 10% by weight, relative to the total weight of the composition.

### (Amino Acid Surfactant)

The composition according to the present invention may comprise (e) at least one amino acid surfactant. A single type of amino acid surfactant may be used, or two or more different types of amino acid surfactants may be used in combination.

The (e) amino acid surfactant is different from the (d) amphoteric surfactant.

The (e) amino acid surfactant is an anionic surfactant based on an amino acid or derivatives thereof. Typically, the (a) amino acid surfactant is an anionic surfactant comprising at least one amino moiety and at least one carboxylic acid moiety which is in the form of a carboxylate. The amino acid surfactant may have two or more amino moieties and/or two or more carboxylic acid moieties which are in the form of carboxylates.

The (e) amino acid surfactant may preferably be selected from amino acid derivatives. The amino acid derivative may more preferably be selected from salts of amino acids and N-acylated amino acids, for example alkali metal salts and alkali earth metal salts of amino acids and N-acylated amino acids, such as sodium salts, potassium salts, magnesium salts, and calcium salts of amino acids and N-acylated amino acids.

The acyl group which forms the N-acyl moiety of the amino acid derivatives may be a C₄-C₂₆ acyl group, preferably a C₆-C₂₄ acyl group, and more preferably a C₈-C₂₂ acyl group.

The (e) amino acid surfactant may be represented by the following chemical formula (VIII):

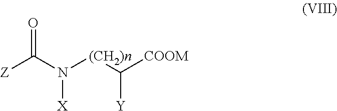

wherein:
Z represents a saturated or unsaturated, linear or branched hydrocarbon group having 8 to 22 carbon atoms,
X is a hydrogen atom or a methyl group,
n is 0 or 1,
Y is selected from a hydrogen atom, —CH₃, —CH(CH₃)₂, —CH₂CH(CH₃)₂, —CH(CH₃)CH₂CH₃, —CH₂C₆H₅, —CH₂C₂H₄OH, —CH₂OH, —CH(OH)CH₃, —(CH₂)₄NH₂, —(CH₂)₃NHC(NH)NH₂, —CH₂C(O)OM, —(CH₂)₂C(O)OH, and —(CH₂)₂C(O)OM, and
M is a salt-forming cation, wherein COO is a counter-anion, such as sodium ion, potassium ion, and ammonium ion.

According to a preferred embodiment of the present invention, in the above formula (VIII):
Z represents a saturated or unsaturated, linear C₈ to C₁₈ alkyl group, in particular a cocoyl group,
X is a hydrogen atom,
n is 0,
Y is a hydrogen atom or —(CH₂)₂C(O)OM, and
M is a salt-forming cation, wherein COO is a counter-anion, such as sodium ion, potassium ion, and ammonium ion.

The (e) amino acid surfactant may be derived from a carboxylate salt of amino acid wherein the amine group situated on the α-carbon or β-carbon of an amino acid salt is acylated with a fatty acid derivative.

The carboxylate salts of these amino acids can be formed by conventional means such as by neutralization of the respective amino acid with a base. The amine group situated on the α-carbon or β-carbon of the neutralized amino acid is acylated with a fatty acid halide (acyl halide) in the presence of a base via a well-known Schotten-Baumann reaction giving the amide, thus forming the desired surfactant reaction product, i.e. the (e) amino acid surfactant.

Suitable acyl halides for acylation of the amino acid carboxylate salt include acyl chlorides, bromides, fluorides, and iodides. The acyl halides can be prepared by reacting a saturated or unsaturated, linear or branched fatty acid with a thionyl halide (bromide, chloride, fluoride, and iodide). Representative acyl halides include but are not limited to the acyl chlorides selected from decanoyl chloride, dodecanoyl chloride (lauroyl chloride), cocoyl chloride (coconut oil derived fatty acid chlorides) tetradecanoyl chloride (myristoyl chloride), hexadecanoyl chloride (palmitoyl chloride), octadecanoyl chloride (stearoyl chloride), 9-octadecenoyl chloride (oleoyl chloride), eicosanoyl chloride (arachidoyl chloride), docosanoyl chloride (behenoyl chloride), and any mixture thereof. Other acyl halides include the bromides, fluorides and iodides of the foregoing fatty acids. A method for preparing acyl halides as well as an alternative method for acylating amino acids is set forth in US Patent Application Publication No. 2008/0200704, published on Aug. 21, 2008, which application is incorporated herein by reference.

The (e) amino acid surfactant may even more preferably be selected from the group consisting of glutamates, N-acylated glutamates, aspartates, N-acylated aspartates, and salts thereof.

Examples of the (e) amino acid surfactant include, but are not limited to:
sarcosinates, such as N-acyl sarcosinates (salts), e.g., sodium lauroyl sarcosinate, sold under the name Nikkol Sarcosinate™ LN by Nikko Chemicals or sold under the name Pureact™ LSR by Innospec Performance Chemicals Europe Limited; sodium myristoyl sarcosinate, sold under the name Nikkol Sarcosinate™ MN by Nikko Chemicals; and sodium palmitoyl sarcosinate, sold under the name Nikkol Sarcosinate™ PN by Nikko Chemicals;
alaninates, such as N-acyl alaninates (salts), e.g., sodium N-lauroyl-N-methylamidopropionate, sold under the name Sodium Nikkol Alaninate™ LN 30 by Nikko Chemicals or sold under the name Alanone® ALE by Kawaken; sodium cocoyl alaninates, sold under the name Amilite® ACS-12 by Ajinomoto; and triethanolamine N-lauroyl-N-methylalanine, sold under the name Alanone® ALTA by Kawaken;
glutamates, such as N-acyl glutamates (salts), e.g., sodium stearoyl glutamate, sold under the name Eumulgin® SR by BASF Japan; sodium lauroyl glutamate, sold under the name Plantapon® Amino SLG-P by BASF Japan; triethanolamine monococoyl glutamate, sold under the name Amisoft® CT-12 by Ajinomoto; triethanolamine lauroyl glutamate, sold under the name Amisoft® LT-12 by Ajinomoto; disodium stearoyl glutamate, sold under the name Amisoft® HS-21P by Ajinomoto; sodium cocoyl glutamate, sold under the name Plantapon® Amino SF-N by BASF Japan; disodium cocoyl glutamate, sold under the name Plantapon® Amino SCG-L by BASF Japan; and disodium/sodium cocoyl glutamate, sold under the name Amisoft® CS-22 by Ajinomoto;
aspartates, such as N-acyl aspartates (salts), e.g., disodium N-lauroyl aspartate, sold under the name AminoFoamer® FLMS-P1 by Asahi Kasei; and a mixture of triethanolamine N-lauroyl aspartate and triethanolamine N-myristoyl aspartate, sold under the name AminoFoamer® FCMT-L by Asahi Kasei; and
glycinates, such as N-acyl glycinates (salts), e.g., sodium N-cocoyl glycinate, sold under the name Amilite® GCS-12K; and potassium N-cocoyl glycinate, sold under the name Amilite® GCK-12K by Ajinomoto.

It is preferable that the (e) amino acid surfactant be selected from glutamates, preferably N-acyl glutamates, and more preferably sodium cocoyl glutamate, disodium cocoyl glutamate and a mixture thereof.

The amount of the (e) amino acid surfactant(s) in the composition according to the present invention may be 0.01% by weight or more, preferably 0.1% by weight or more, and more preferably 1% by weight or more, relative to the total weight of the composition.

The amount of the (e) amino acid surfactant(s) in the composition according to the present invention may be 20% by weight or less, preferably 15% by weight or less, and more preferably 10% by weight or less, relative to the total weight of the composition.

The amount of the (e) amino acid surfactant(s) in the composition according to the present invention may be from 0.01% to 20% by weight, preferably from 0.1% to 15% by weight, and more preferably from 1% to 10% by weight, relative to the total weight of the composition.

### (Monovalent Non-Polymeric Acid or Salt Thereof)

The composition according to the present invention may comprise (f) at least one monovalent non-polymeric acid or a salt thereof. A single type of monovalent non-polymeric acid or a salt thereof may be used, or two or more different types of monovalent non-polymeric acids or salts thereof may be used in combination.

The (f) monovalent non-polymeric acid or a salt thereof is different from the (e) amino acid surfactant.

The term “non-polymeric” here means that the acid is not obtained by polymerizing two or more monomers. Therefore, the non-polymeric acid does not correspond to an acid obtained by polymerizing two or more monomers such as polyacrylic acids.

The term “salt” here means a salt formed by addition of suitable base(s) to the monovalent non-polymeric acid, which may be obtained from a reaction with the monovalent non-polymeric acid with the base(s) according to methods known to those skilled in the art. As the salt, mention may be made of metal salts, for example salts with alkaline metal such as Na and K, and salts with alkaline earth metal such as Mg and Ca, and ammonium salts.

It is preferable that the molecular weight of the (f) monovalent non-polymeric acid or salt thereof be less than 1,000, preferably 500 or less, and more preferably 200 or less.

The (f) monovalent non-polymeric acid or a salt thereof can be included in the aqueous phase formed by the (c) water, if present. The (f) monovalent non-polymeric acid or a salt thereof may promote the dissolution of the (a) cationic polymer in the (c) water, if present.

The (f) monovalent non-polymeric acid has a single acid group which may be selected from the group consisting of a carboxylic group, a sulfuric group, a sulfonic group, a phosphoric group, a phosphonic group, and a mixture thereof.

The (f) monovalent non-polymeric acid or a salt thereof may be selected from monovalent organic or inorganic acids and salts thereof.

It is preferable that the (f) monovalent non-polymeric acid be a monovalent organic acid, and more preferably a monovalent non-polymeric carboxylic acid.

The monovalent non-polymeric carboxylic acid may be selected from hydroxy acids, and preferably alpha-hydroxy acids and beta-hydroxy acids. As the alpha-hydroxy acids, mention may be made of, for example, lactic acid and glycolic acid. As the beta-hydroxy acids, mention may be made of, for example, salicylic acid.

Thus, the monovalent non-polymeric acid may be a monovalent non-polymeric organic acid, preferably a monovalent non-polymeric carboxylic acid, and more preferably a monovalent hydroxy acid such as lactic acid and salicylic acid.

The amount of the (f) monovalent non-polymeric acid(s) or salt(s) thereof in the composition according to the present invention may be 0.01% by weight or more, preferably 0.05% by weight or more, and more preferably 0.1% by weight or more, relative to the total weight of the composition.

The amount of the (f) monovalent non-polymeric acid(s) or salt(s) thereof in the composition according to the present invention may be 15% by weight or less, preferably 10% by weight or less, and more preferably 5% by weight or less, relative to the total weight of the composition.

The amount of the (f) monovalent non-polymeric acid(s) or salt(s) thereof in the composition according to the present invention may be from 0.01% to 15% by weight, preferably from 0.05% to 10% by weight, and more preferably from 0.1% to 5% by weight, relative to the total weight of the composition.

### (Optional Ingredient)

The composition according to the present invention may comprise, in addition to the aforementioned ingredients, optional ingredient(s) typically employed in cosmetics, specifically, surfactants/emulsifiers other than the (b) glycolipid, (d) amphoteric surfactant, and the (e) amino acid surfactants, such as taurates (e.g., sodium cocoyl taurate, sodium methyl taurate and sodium lauroyl taurate); hydrophilic or lipophilic thickeners derived from, for example, synthetic thickeners; volatile or non-volatile organic solvents such as ethanol; polyols such as glycerin, pentylene glycol, dipropyleneglycol, propylene glycol, butylene glycol, propanediol and sorbitol; anionic polymers; amphoteric polymers; nonionic polymers such as beta-glucan; silicones and silicone derivatives; natural extracts derived from animals or vegetables other than the (a) cationic polymer; waxes; preservatives such as caprylyl glycol, phenoxyethanol; antioxidants such as tocopherol; salts such as NaCl; and the like, within a range which does not impair the effects of the present invention.

The composition according to the present invention may comprise the above optional ingredient(s) in an amount of from 0.01% to 30% by weight, preferably from 0.05% to 20% by weight, and more preferably from 0.1% to 10% by weight, relative to the total weight of the composition.

The composition according to the present invention may comprise at least one oil. Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

However, the amount of the oil(s) in the composition according to the present invention may be limited. Thus, the amount of oil(s) in the composition according to the present invention may be less than 5% by weight, relative to the total weight of the composition. It may particularly be preferable that the composition according to the present invention includes no oil.

The amount of the synthetic thickener(s) in the composition according to the present invention may be less than 3% by weight, relative to the total weight of the composition. It may particularly be preferable that the composition according to the present invention includes no synthetic thickener.

The amount of the anionic polymer or the amphoteric polymer in the composition according to the present invention may be less than 1% by weight, preferably less than 0.1% by weight, and more preferably less than 0.01% by weight, relative to the total weight of the composition. It is particularly preferable that the composition according to the present invention includes no anionic or amphoteric polymer.

[pH]

The pH of the composition according to the present invention may be from 4 to 8, preferably from 4.5 to 7.5, and more preferably from 5 to 7.

The pH of the composition according to the present invention may be adjusted by adding (f) monovalent non-polymeric acid or a salt thereof, or at least one alkaline agent and/or at least one acid, other than the (f) monovalent non-polymeric acid or a salt thereof. The pH of the composition according to the present invention may also be adjusted by adding at least one buffering agent.

### [Preparation]

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention.

The composition according to the present invention can be prepared by simple or easy mixing with a conventional mixing means such as a stirrer and a homogenizer. Also, heating may not be necessary. Therefore, the process for preparing the composition according to the present invention may be environmentally friendly.

### [Form]

The composition according to the present invention may be present in any form.

For example, the composition according to the present invention can be in the form of a solution or a gel.

In another embodiment, the composition according to the present invention can be in the form of a solution. In this embodiment, the composition according to the present invention may be packaged with an auto foam pump which enables consumers to use the composition as a foam.

The composition according to the present invention, before being diluted, can have a transparent or translucent appearance, preferably a transparent appearance.

The transparency may be measured by measuring the turbidity (for example, with 2100Q Portable Turbidimeter from HACH). The turbidity of the composition according to the present invention may be below 400 NTU (translucent), preferably below 350 NTU, more preferably below 300 NTU (transparent).

### [Cosmetic Application]

The composition according to the present invention may be intended to be used as a cosmetic composition. Thus, the cosmetic composition according to the present invention may be intended for application onto a keratin substance. Keratin substance here means a material containing keratin as a main constituent element, and examples thereof include the skin, scalp, nails, lips, hair, and the like. Thus, it is preferable that the cosmetic composition according to the present invention be used for a cosmetic process for the keratin substance, in particular skin.

Thus, the cosmetic composition according to the present invention may be a skin cosmetic composition, preferably a skin care composition, and more preferably a face care composition.

In particular, the composition according to the present invention is useful for cleansing. Thus, it is preferable that the composition according to the present invention be a cleansing composition, more preferably a cleansing composition for skin, and even more preferably a cleansing composition for face.

### [Cosmetic Process and Use]

The present invention also relates to:
a cosmetic process for a keratin substance such as skin, comprising applying to the keratin substance the composition according to the present invention; and optionally removing the composition from the keratin substance, or
a use of the composition according to the present invention for caring for or cleansing a keratin substance such as skin.

The cosmetic process here means a non-therapeutic cosmetic method, such as for a cosmetic method for caring for or cleansing the surface of a keratin substance such as skin.

The strep of removing in the cosmetic process according to the present invention can be performed by, for example, rinsing off with water the composition according to the present invention from a keratin substance such as skin.

If the step of removing is not performed, the cosmetic process according to the present invention can be useful for caring for a keratin substance such as skin, because the (b) glycolipids can provide the keratin substance with anti-inflammation, anti-allergic, or anti-bacterial properties, which would be useful for, in particular, caring for skin with acne and sensitive skin.

If the step of removing is performed, the cosmetic process according to the present invention can be useful for cleansing a keratin substance such as skin, because the (b) glycolipids can function as a surfactant.

If the composition according to the present invention is diluted with water, the composition according to the present invention can cause precipitation.

Thus, before or after the step of applying the composition according to the present invention to a keratin substance such as skin, if the keratin substance is wet with water, the composition according to the present invention can cause precipitates on the keratin substance.

On the other hand, before the step of removing the composition according to the present invention from a keratin substance such as skin, if the keratin substance is wet with water, the composition according to the present invention can also cause precipitation on the keratin substance.

Accordingly, it is preferable that a keratin substance such as skin be wet with water before or after the step of applying the composition according to the present invention to the keratin substance, or before removing the composition according to the present invention from the keratin substance.

The precipitates include the (b) glycolipids, and therefore, it can provide a keratin substance such as skin with various benefits such as anti-inflammation, anti-allergic, or anti-bacterial properties, which would be useful for, in particular, skin with acne and sensitive skin.

Furthermore, the precipitates include (b) glycolipids which can function as surfactants. Therefore, the precipitates can effectively be used for cleaning a keratin substance such as skin, by further rinsing off with water the precipitates from the keratin substance, which would be preferable for cleansing the keratin substance, in particular for removing makeup from the keratin substance such as skin.

The present invention may also relate to a use of (a) at least one cationic polymer selected from chitosans with a molecular weight of less than 20,000, preferably less than 15,000 and more preferably less than 10,000,
in a composition comprising
(b) at least one glycolipid,
in order to provide precipitates including the (b) glycolipid when diluting the composition with water.

The above explanations regarding the (a) cationic polymer, the (b) glycolipid, and the (c) water for the composition according to the present invention, can apply to those in the above use.

### EXAMPLES

The present invention will be described in a more detailed manner by way of examples. However, they should not be construed as limiting the scope of the present invention.

### Examples 1-7 and Comparative Examples 1-5

### [Preparations]

Each of the compositions according to Examples 1-7 and Comparative Examples 1-5 was prepared by mixing the ingredients shown in Table 1. The numerical values for the amounts of the ingredients in Table 1 are all based on “% by weight” as active ingredients.

TABLE 1

| | | | | | | Comp. | Comp. | Comp. | Comp. | Comp. | | |
|---|---|---|---|---|---|---|---|---|---|---|---|---|
| | Ex. 1 | Ex. 2 | Ex. 3 | Ex. 4 | Ex. 5 | Ex. 1 | Ex. 2 | Ex. 3 | Ex. 4 | Ex. 5 | Ex. 6 | Ex. 7 |
| Water | qsp100 | qsp100 | qsp100 | qsp100 | qsp100 | qsp100 | qsp100 | qsp100 | qsp100 | qsp100 | qsp100 | qsp100 |
| Chitosan (MW ≤3000) | 1 | 1 | 1 | 1 | 1 | 1 | 1 | 1 | — | — | 0.6 | 3 |
| Chitosan (MW = 25,000) | — | — | — | — | — | — | — | — | 1 | — | — | — |
| Chitosan (MW = 115,000) | — | — | — | — | — | — | — | — | — | 1 | — | — |
| Lactic Acid | 1.4 | — | 0.9 | 0.9 | 0.9 | 1.35 | — | 1.35 | 0.9 | 0.9 | 0.9 | 0.9 |
| Glycolipids | 1.5 | 1.5 | 1.5 | 3 | 3 | — | — | — | 1.5 | 1.5 | 1.5 | 1.5 |
| Cocamidopropylbetaine | 1.2 | 2.3 | 2.3 | 1.2 | 2.3 | 1.14 | 2.28 | 2.28 | 2.3 | 2.3 | — | 5.2 |
| Disodium/Sodium Cocoyl | 5.3 | — | 3.6 | 4.8 | 3.8 | 6.75 | — | 5.025 | 3.6 | 3.6 | 3.2 | 4.5 |
| Glutamide | | | | | | | | | | | | |
| pH | 5.5 | 5.3 | 5.5 | 5.6 | 5.5 | 5.5 | 5.5 | 5.5 | 5.5 | 5.5 | 5.5 | 5.5 |
| Dilution Test | Good | Good | Good | Good | Good | Poor | Poor | Poor | Very | Very | Good | Very |
| | | | | | | | | | Poor | Poor | | Good |

### [Evaluation]

### (Dilution Test)

Each of the compositions according to Examples 1-7 and Comparative Examples 1-5 was diluted with water to have 5 times volume at room temperature (25° C.).

The dilution test simulates the use of the composition according to the present invention under wet conditions such as those in a bath room.

The precipitation in each composition was visually evaluated in accordance with the following criteria.
Very Good: A lot of precipitation was observed after dilution
Good: Precipitation was observed after dilution
Poor: No precipitation was observed after dilution
Very Poor: Precipitation was observed before dilution

The results are shown in Table 1.

The more the precipitation is caused, the better the composition according to the present invention can provide effects based on the precipitation.

### (Summary)

Examples 1-6 show that the compositions according to Examples 1-6 including a low molecular weight chitosan and glycolipids were able to provide precipitates when being diluted with water.

Example 7 shows that the compositions according to Example 7 was able to provide a lot of precipitates when being diluted with water, due to the use of a relatively large amount of a low molecular weight chitosan.

Comparative Examples 1-3 show that the compositions according to Comparative Examples 1-3 including no glycolipids could not provide any precipitates although they include a low molecular weight chitosan.

Comparative Examples 4 and 5 show that the compositions according to Comparative Examples 4 and 6 including chitosan with a molecular weight of 20,000 or more caused precipitation before being diluted with water. The precipitates before being diluted with water is not preferable because of the non-uniformity or non-homogeneity of the compositions, for example, during storage.

1. A composition, comprising:
(a) at least one cationic polymer selected from chitosans; and
(b) at least one glycolipid,
wherein
the (a) at least one cationic polymer has a molecular weight of less than 20,000.

2. The composition according to claim 1, wherein the amount of the (a) at least one cationic polymer in the composition is from 0.01% to 15% by weight relative to the total weight of the composition.

3. The composition according to claim 1, wherein the (b) at least one glycolipid is selected from rhamnolipids.

4. The composition according to claim 1, wherein the amount of the (b) at least one glycolipid in the composition is from 0.01% to 15% by weight relative to the total weight of the composition.

5. The composition according to claim 1, wherein the composition further comprises (c) water.

6. The composition according to claim 1, wherein the composition further comprises (d) at least one amphoteric surfactant.

7. The composition according to claim 6, wherein the amount of the (d) at least one amphoteric surfactant in the composition is from 0.01% to 20% by weight relative to the total weight of the composition.

8. The composition according to claim 1, wherein the composition further comprises (e) at least one amino acid surfactant.

9. The composition according to claim 8, wherein the amount of the (e) at least one amino acid surfactant in the composition is from 0.01% to 20% by weight relative to the total weight of the composition.

10. The composition according to claim 1, wherein the composition further comprises (f) at least one monovalent non-polymeric acid or a salt thereof.

11. The composition according to claim 10, wherein the amount of the (f) at least one monovalent non-polymeric acid or a salt thereof in the composition is from 0.01% to 15% by weight relative to the total weight of the composition.

12. The composition according to claim 1, wherein the pH of the composition is from 4 to 8.

13. The composition according to claim 1, which is a cleansing composition.

14. A cosmetic process for a keratin substance, comprising:
applying to the keratin substance the composition according to claim 1; and
optionally removing the composition from the keratin substance.

15. A method, comprising:
combining (a) at least one cationic polymer selected from chitosans with a molecular weight of less than 20,000 with
a composition comprising (b) at least one glycolipid,
in order to provide precipitates including the (b) at least one glycolipid when diluting the composition with water.
